(19) United States
(12) Patent Application Publication
(10) Pub. No.: US 2021/0027003 A1
(43) Pub. Date: Jan. 28, 2021

(54) METHOD AND DEVICE FOR MODELLING AND FATIGUE STRENGTH ASSESSMENT OF WELD SEAMS BETWEEN MECHANICAL PARTS

(71) Applicant: 7tech GmbH, Eggenburg (AT)

(72) Inventor: Christoph Schlegel, Eggenburg (AT)

(21) Appl. No.: 16/938,029

(22) Filed: Jul. 24, 2020

(30) Foreign Application Priority Data
Jul. 25, 2019 (EP) 19 188 469.1

(51) Int. Cl. G06F 30/23; G01N 33/207

(52) U.S. Cl. CPC G06F 30/23; G06F 2111/10; G01N 33/207

(57) ABSTRACT

A method for modelling and fatigue strength assessment of weld seams between mechanical parts includes providing a finite element model for an assembly, in which a first finite element mesh for a mechanical part, a second mesh for a second mechanical part, and a third mesh for a weld seam joining the mechanical parts comprising a number of notches are generated. The third mesh has fewer than 20 finite elements in cross-section, the notches are modelled sharp-edged, and the distribution of the finite elements follows a defined mesh pattern. The method includes calculating the finite element model. Result values of the finite elements and nodes are provided from the defined mesh pattern of the third mesh. The method includes applying an effective notch stress prediction algorithm matched to the defined mesh pattern to predict occurring notch stresses in the notches using the provided result values as input parameters.

### FIELD

The present invention is directed to a computer-implemented method and a computer-implemented device for modelling and fatigue strength assessment of weld seams between mechanical parts of an assembly with the aid of a finite element method.

### BACKGROUND

The technical field of the invention concerns the creation of a model and fatigue strength assessment of weld seams between mechanical parts of an assembly with the aid of a finite element method.

CAE analyses (CAE, computer-aided engineering) performed by a computer are widely used to simulate and evaluate the usability of technical structures. A common method of CAE analyses are finite element analyses. Mechanical parts are meshed with finite elements to calculate deformations or mechanical stresses under given loads. This can also be used to evaluate the strength of a structure, for example.

If Finite Element Models (FE Models) are calculated that contain welds, the calculated stresses in the welds do not directly provide information about their structural (fatigue) strength. On the one hand, the FE models usually do not contain the notches (see FIG. 1) of the relevant weld seam accurately enough, on the other hand, the microstructure of the material surrounding the weld seam is changed after melting and cooling and cannot be assessed like the parent material.

For this purpose, FIG. 1 shows a cross-section of a welded assembly 10. The mechanical parts 2 and 3 of assembly 10 are connected by welds 4a and 4b. For the fatigue strength assessment of welds 4a, 4b with the Effective Notch Stress method, the notches 5a, 5b, 5c are rounded with a radius defined in corresponding codes of practice.

There are a number of standards that describe supplementary methods for carrying out weld seam assessment on the basis of the FE results. The methods are divided in particular into the following three groups A, B and C:

Group A:

With Nominal Stress methods, forces and moments on the weld seams are evaluated and thus nominal stresses are calculated for a weld cross-section. These nominal stresses must then be compared with permissible values according to a notch case class (FAT class), which the user must select manually. This requires a certain know-how and leaves room for interpretation from case to case, which leads to discussions and fluctuating results. Special cases of weld seam constellations are often not included in standard notch class catalogs.

Group B:

FIG. 2 shows Structural Hot Spot Stress methods. For this purpose, the welding seams has to be modelled in a certain way in the FE-model and continuously connected within the FE-mesh (without FE contact elements). For the assessment, stresses of defined supporting points in the parent material (next to the weld seam) are evaluated and extrapolated into the weld seam notch. These stresses are then again compared with notch case classes (FAT classes) depending on the type of weld seam. This means considerable effort in model creation and evaluation. And it again requires a certain amount of know-how from the user and sometimes leaves room for subjective interpretation when choosing the right notch case class. The illustrations in FIG. 2 are taken from the IIW guideline according to reference [1].

Group C:

The most accurate and generally applicable Effective Notch Stress methods are shown in FIG. 3. In FIG. 3, reference sign 30 shows a finely meshed FE model of an assembly with two welded mechanical parts 2, 3, the respective weld 4a, 4b having rounded notches 5a, 5b, 5c. For reasons of simplicity, the notches are marked with the reference signs 5a, 5b, 5c only for the right-hand weld 4a. The notches 5a, 5b, 5c must be modelled with a standardized radius and very finely meshed. This also means considerable modelling and calculation effort. The assessment is slightly easier here, as the notch stresses are compared with fixed, standardized permissible values (e.g. FAT 225). This method is suitable for any weld seam constellation, it leaves little room for subjective interpretation and is therefore easier to use. However, since very fine meshing and thus a very large calculation effort is required, the Effective Notch Stress method is typically only used only for small, very local geometry details in so-called submodels. For large models with a large number of weld seams, the “Effective Notch Stress” method cannot be applied economically across the entire FE model.

Although Effective Notch Stress methods are the most universally applicable and accurate methods, they also require the most calculation effort. They require the least experience and know-how from the user, as there is no need to select weld seam notches or fatigue classes from catalogs.

All conventional methods require correspondingly targeted modeling and meshing. Nominal Stress and Structural Hot Spot Stress methods use FE models with fewer nodes and therefore less computational effort, but require more user input and experience and are less accurate than Effective Notch Stress methods. The latter are very accurate and straightforward to use, but also require a lot of computation.

Information about conventional methods for modelling and fatigue strength assessment of welds between mechanical parts of an assembly can be found in references [1] to [9].

### SUMMARY

Based on this background, on object of the present invention is to improve the modelling and fatigue strength assessment of welds between mechanical parts of an assembly.

According to a first aspect, a computer-implemented method for model generation and fatigue strength assessment of weld seams between mechanical parts of an assembly using a finite element method is proposed. The method comprises the following steps:

a) providing a finite element model for the assembly, in which a first finite element mesh for a first mechanical part, a separate second finite element mesh for a second mechanical part and a third finite element mesh for a weld seam joining the first mechanical part and the second mechanical part comprising a number of notches are generated, wherein the third finite element mesh has a number of less than 20 finite elements in cross-section, the notches of the weld seam are sharp-edged modelled and the distribution of the finite elements follows a defined mesh pattern,
b) calculating the finite element model, wherein result values of the finite elements and nodes are provided from the defined mesh pattern of the third finite element mesh for the weld seam, and
c) applying an effective notch stress prediction algorithm matched to the defined mesh pattern of the third finite element mesh to predict occurring notch stresses in the notches (in a rounded state) using the provided result values as input parameters.

This facilitates and accelerates the calculations and fatigue strength assessment of welds in finite element models using the Effective Notch Stress method. This makes it possible to model the welds sharp-edged and to mesh them relatively coarsely and still obtain a good prognosis for the effective notch stresses with which a fatigue strength assessment can subsequently be carried out easily. This speeds up the calculation and evaluation process considerably and also allows an effective notch stress prediction algorithm to be applied to FE models with a large number of welds. The modelling and evaluation of the welds can advantageously be well automated and executed by software programs.

Since, with the present computer-implemented method, the Effective Notch Stress method can be used also for complex FE models, the user needs less know-how than with conventional methods for complex FE models, which require the selection of a notch case class (FAT class). Furthermore, the user can intuitively check the exact type and geometric dimensions of the welds by means of volume meshing and thus avoid errors (compared with FE shell modelling methods). Due to the fact that the Effective Notch Stress method is used, the present method is also not limited to a restricted group of weld seam types from a notch case catalog, but can analyze any welding constellation.

When categorizing weld fatigue strength assessment methods, the proposed method can be placed between the existing Structural Hot Spot Stress methods and the “Effective Notch Stress” methods. Due to the smaller number of FE nodes, the proposed method combines the lower calculation effort of the “Structural Hot Spot Stress” methods with the more general applicability and the more accurate geometric modelling and easier evaluation of the Effective Notch Stress methods.

The third finite element mesh has a number of 1 to 20 finite elements in the cross-section. A small number of elements leads to lower calculation accuracy, but also advantageously to shorter calculation times. A larger number of elements and especially smaller elements near the weld notches lead to more accurate results with greater calculation effort and time. The method can therefore be trimmed more in the direction of shorter calculation time or higher accuracy, as required.

The finite element is in particular a solid element. The finite elements of the weld seams are in 3D models especially 3D volume elements and in 2D models 2D elements and complete the FE model of the unwelded mechanical parts.

Examples of mechanical parts include thin-walled parts, such as sheet metal or profiles, or thick-walled or bulky parts, such as castings.

According to an embodiment, the first finite element mesh and the third finite element mesh are coupled with a first number of coupling elements and the second finite element mesh and the third finite element mesh are coupled with a second number of coupling elements.

By using the coupling elements, no common continuous meshing between the third finite element mesh for the weld seam and the first finite element mesh for the first mechanical part is necessary. Accordingly, no common continuous meshing between the third finite element mesh for the weld seam and the second finite element mesh for the second mechanical part is necessary. Therefore, the third finite element mesh can be subsequently added to an existing and unwelded component mesh comprising the first finite element mesh and the second finite element mesh. Between the finite element meshes for the weld seam and for the components no common nodes are necessary. This has the advantage of reducing the effort required for meshing. The third finite element mesh for the weld seam can also be integrated subsequently.

According to a further embodiment, step b) is formed by:

b1) calculating the finite element model, and
b2) evaluating the result values of the finite elements and the nodes of the defined mesh pattern for the weld seam on the basis of the calculated finite element model.

According to a further embodiment, in step b2) result values are evaluated exclusively within the finite elements and the nodes of the third finite element mesh for the weld seam.

According to a further embodiment, the result values which are evaluated in step b2) include
stress results,
reaction force results,
geometry parameters, and/or
material parameters.

According to a further embodiment, the result values which are evaluated in step b2) consist of
stress results,
reaction force results,
geometry parameters, and/or
material parameters.

According to a further embodiment, the effective notch stress prediction algorithm is trained with a plurality of weld seam parameter variants using the defined mesh pattern before step c) is applied.

Each weld seam constellation preferably has different geometric dimensions (parameters) of the components and the weld seam as well as different loads (parameters) and represents a design point in the parameter space. From each design point, preferably firstly the present modelling method and secondly a variant with standard notch rounding radius and very fine meshing as in FIG. 3 is calculated. The second model provides the reference results (target values) of the weld seam effective notch stresses and the first model provides the input data for the effective notch stress prediction algorithm. Thus, the effective notch stress prediction algorithm is fitted (trained) to the existing meshing pattern, the given notch radius and the existing modelling method of the first model. The algorithm trained in this way can then be applied to productive FE models to predict weld seam effective notch stresses.

Although the effective notch stress prediction algorithm requires a certain amount of effort in its creation (during fitting or training), it is very efficient in the subsequent application and requires only very little computing time.

According to a further embodiment, in step c) a plurality of parameters of the notches are predicted by the effective notch stress prediction algorithm.

According to a further embodiment, these parameters include
normal stresses,
shear stresses,
Von Mises equivalent stresses, and/or
radial, tangential and/or axial stress components
   in the notch radius of the respective notch.

According to a further embodiment, the predicted notch stresses are then used to perform fatigue strength assessment of the assembly.

According to second aspect, a computer program product is proposed, which, on a program-controlled device, initiates the execution of the method described above.

A computer program product, such as a computer program resource, may be provided or delivered, for example, as a storage medium, such as a memory card, USB stick, CD-ROM, DVD, or in the form of a downloadable file from a server on a network. This can be done, for example, in a wireless communication network by transferring a corresponding file with the computer program product or computer program resource.

According to a third aspect, a computer-implemented device for modelling and fatigue strength assessment of weld seams between mechanical parts of an assembly using a finite element method is proposed. The device comprises:
a first unit for providing a finite element model for the assembly, in which a first finite element mesh for a first mechanical part, a separate second finite element mesh for a second mechanical part and a third finite element mesh for a weld joining the first mechanical part and the second mechanical part comprising a number of notches are generated, wherein the third finite element mesh has a number of less than 20 finite elements in cross-section, wherein the notches of the weld seam are sharp-edged modelled and the distribution of the finite elements follows a defined mesh pattern,
a second unit for calculating the finite element model, wherein result values of the finite elements and nodes are provided from the defined mesh pattern of the third finite element mesh for the weld seam, and
a third unit for applying an effective notch stress prediction algorithm matched to the defined mesh pattern of the third finite element mesh to predict occurring effective notch stresses in the notches using the provided result value input parameters.

The respective unit can be implemented in hardware and/or in software. In the case of a hardware implementation, the unit can be designed as a device or as part of a device, for example as a computer or as a microprocessor. In a software implementation, the unit may be a computer program product, a function, a routine, part of a program code or an executable object.

The embodiments and features described for the proposed method apply accordingly to the proposed device.

Further possible implementations of the invention also include combinations of features or embodiments not explicitly mentioned before or in the following regarding the examples of execution. In doing so, the skilled person will also add individual aspects as improvements or additions to the respective basic form of the invention.

Further advantageous features and aspects of the invention are the subject of the sub-claims and the examples of implementation of the invention described below. Furthermore, the invention is further explained on the basis of preferred embodiments with reference to the enclosed figures.

### BRIEF DESCRIPTION OF THE DRAWINGS

FIG. 1 shows a cross-section of a welded assembly;

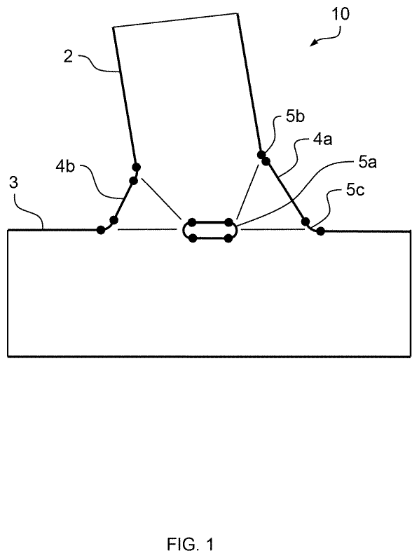

FIG. 2 shows as the first example the Structural Hot Spot Stress method as the state of the art;

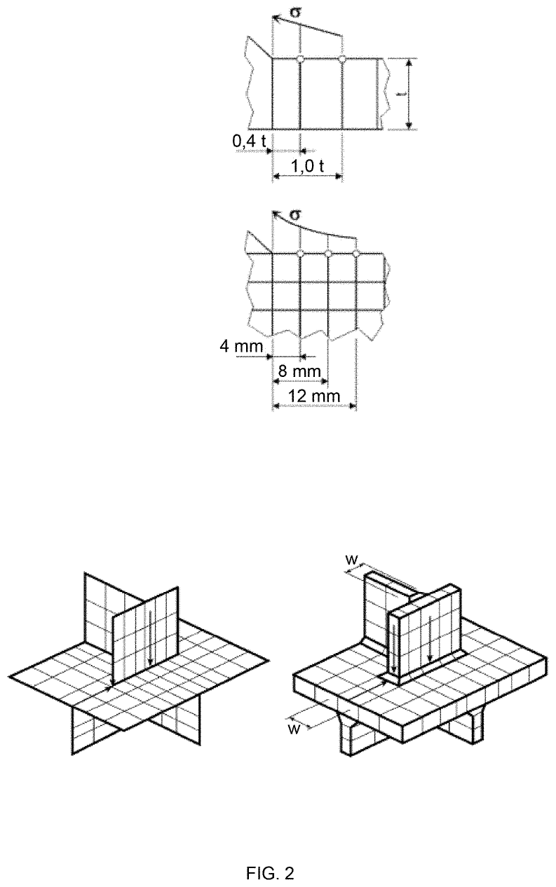

FIG. 3 shows as the first example the Effective Notch Stress method as the state of the art;

FIG. 4 shows the cross-section of a multibody FE model with the weld seam modeled according to the invention;

FIG. 5 shows the cross-section of a multibody FE model with a variant of a weld seam modelled according to the invention;

FIG. 6 shows the cross-section of a multibody FE model with a further variant of a weld seam modelled according to the invention;

FIG. 7 shows cross-sections of FE-models of assemblies with different variants of welds modelled according to invention;

FIG. 8 shows various applications of welds modelled according to the invention;

FIG. 9 shows a schematic flow chart of an execution example of a computer-implemented method for modelling and fatigue strength assessment of welds between mechanical parts of an assembly using a finite element method; and

FIG. 10 shows a schematic block diagram of an embodiment of a computer-implemented device for modelling and fatigue strength assessment of welds between mechanical parts of an assembly using a finite element method.

### DETAILED DESCRIPTION

In the figures, identical or functionally identical elements have been provided with the same reference signs, unless otherwise indicated.

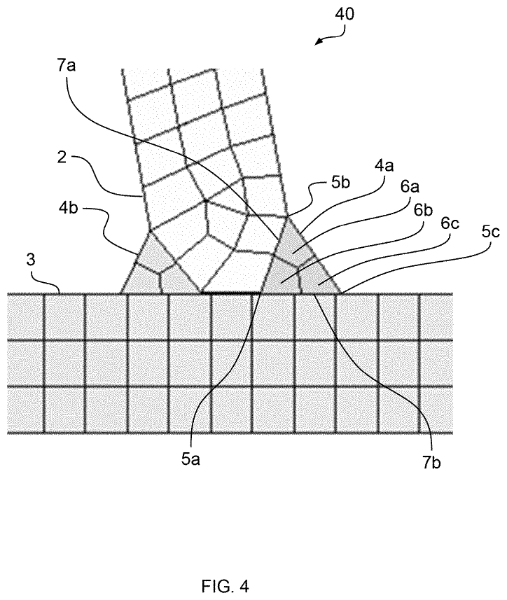

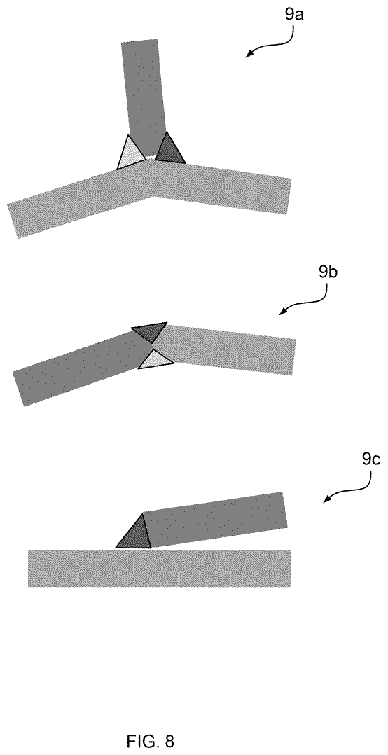

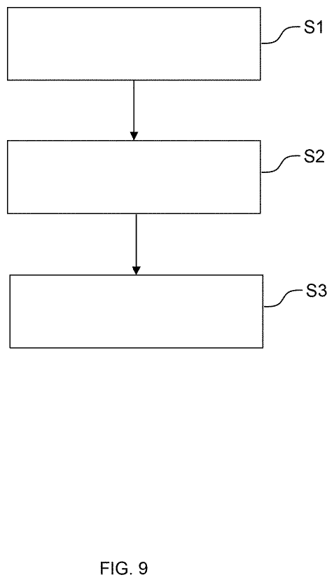

Embodiments for model generation and fatigue strength assessment of welds 4a, 4b between mechanical parts 2, 3 of an assembly are explained with common reference to FIGS. 4 to 9. FIGS. 4 to 7 show examples of FE models of an assembly with a weld seam according to the invention. Furthermore, FIG. 8 shows applications of weld seams modelled according to the invention. Furthermore, FIG. 9 shows an implementation example of a computer-implemented method for model generation and strength evaluation of welds 4a, 4b between mechanical parts 2, 3 of an assembly with the aid of a finite element method.

Starting with FIG. 4, it shows an FE model 40 of a welded assembly. A first mechanical part 2 and a second mechanical part 3 of the assembly are welded by means of two welds 4a, 4b. The notches of weld 4a are marked with the reference signs 5a, 5b, 5c and the finite elements representing weld 4a are marked with the reference signs 6a, 6b, 6c. For reasons of clarity, only the notches and finite elements of weld 4a are marked with reference signs, but not the notches and finite elements of weld 4b.

The finite elements 6a, 6b, 6c are especially designed as 3D volume elements for 3D models and as 2D elements for 2D models and supplement the FE model 40 of the welded component. The components 2, 3 and the welds 4a, 4b can be meshed either with common nodes or independently of each other with separate nodes. A separated, independent meshing has the advantage that the variation of the weld seam geometry is easier to achieve and no changes to the basic model of the mechanical part 2, 3 are necessary.

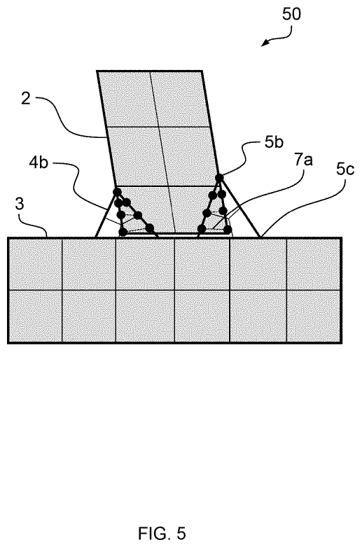

With independent meshing, the weld seam elements 6a, 6b, 6c are connected to mechanical parts 2, 3 with the aid of FE coupling elements 7a, 7b (see also FIG. 5). Examples for coupling elements 7a, 7b include FE contact elements, FE coupling bars or coupling equations.

The independent meshing and connection with FE coupling elements 7a, 7b is made possible, since preferably result values are only evaluated from within the weld seam elements or nodes.

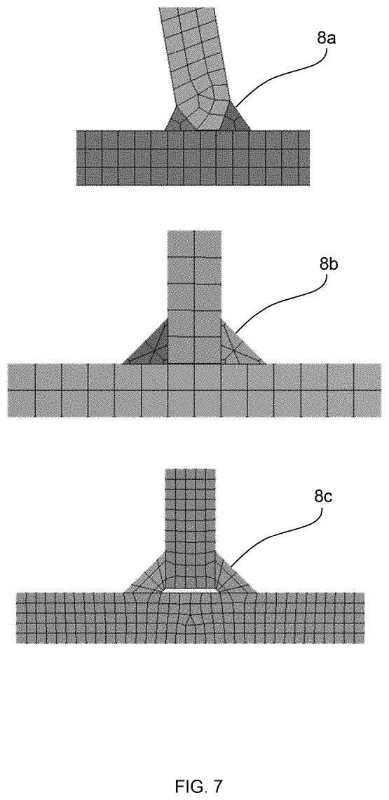

The welds 4a, 4b are meshed with a defined mesh pattern, whereby these mesh patterns are matched to a subsequently used effective notch stress prediction algorithm. The weld seam elements 6a, 6b, 6c preferably have a predefined number, a predefined distribution and a predefined position within the weld seam 4a, 4b. The notches 5a, 5b, 5c of the weld 4a are not rounded but modelled sharp-edged. This allows a relatively coarse meshing and thus saves considerable calculation effort and calculation time. The geometry, the dimensions and the position of the respective weld 4a, 4b are preferably modelled realistically, which is why the stiffness is represented with good accuracy (betters than with FE shell modelling) and the defined mesh pattern is proportionally adapted to the given weld geometry. FIG. 7 shows some examples of structured mesh patterns 8a, 8b, 8c for welds 4a, 4b. It should be noted that the effective notch stress prognosis algorithm used in the following is adapted to the mesh pattern 8a, 8b, 8c used. The creation of a weld seam mesh with defined mesh pattern 8a, 8b, 8c can be carried out automatically using software routines (see method step S1 of FIG. 9).

The FE model of the assembly prepared in this way, including the weld seams 4a, 4b, is then solved using an established FE calculation method and the results are evaluated (see process step S2 of FIG. 9).

A number of parameters of the weld seams 4a, 4b are evaluated and made available to the effective notch stress prognosis algorithm as input data. The parameters can be stresses, strains and/or reaction forces of the weld seam elements 6a, 6b, 6c and nodes. In addition, material and/or geometry parameters such as the dimensions of the weld cross-section, relative position coordinates of individual nodes within the weld cross-section or connection angles of the connected geometry in the individual weld cross-sections and notches can be used.

The effective notch stress prediction algorithm includes in particular metamodels or response surface methods, such as
Global polynomials
Moving leased squares
Kriging
Radial basis functions
Neuronal networks

These models are created (fitted or trained) using:
Regression
Interpolation
Extrapolation

The effective notch stress prediction algorithms are each fitted (trained) to a given weld modelling method with a given mesh pattern. Input data of the effective notch stress prediction algorithm is a relevant subset of the above mentioned parameters. Output data are effective notch stresses and notch stress components for each weld notch per weld cross-section.

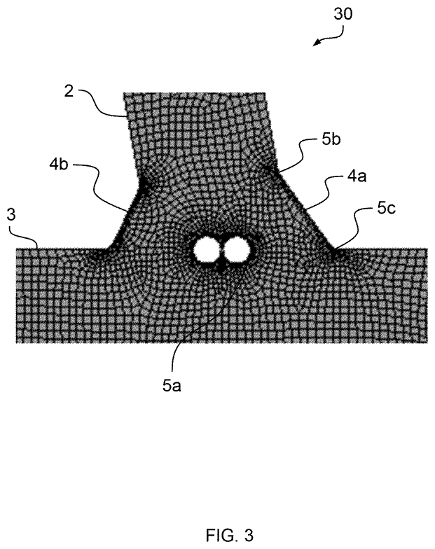

In order to fit (train) the effective notch stress prediction algorithm, preferably a sufficient number of weld seam constellations is calculated. Each weld constellation has different geometric dimensions (parameters) of the components and the weld as well as different loads (parameters) and represents a design point in the parameter space. From each design point, preferably firstly the present modelling method and secondly a variant with a standard notch rounding radius and very fine meshing as shown in FIG. 3 is calculated. The second model provides the reference results (target values) of the weld seam notch stresses and the first model provides the input data for the effective notch stress prediction algorithm. Thus, the effective notch stress prediction algorithm is fitted (trained) to the existing meshing pattern, the given notch radius and the existing modelling method of the first model. The trained algorithm can then be applied to productive FE models to predict weld notch stresses. Even though the prediction accuracy may be slightly lower than with the classical rounded and finely meshed Effective Notch Stress method, the method according to the invention still results in an enormous advantage, since considerably shorter calculation times can be achieved with considerably fewer nodes, or it is only made possible in the first place that “Effective Notch Stress” method can be applied economically to complex finite element models with a high number of weld seams. Without the present method, the number of elements and nodes for Effective Notch Stress calculations on complex models would be too large to be calculated economically.

With the effective notch stresses and notch stress components predicted in this way, a fatigue strength assessment of the weld seam can then be carried out.

This effective notch stress evaluation method is applied to a cross-section of a weld seam, i.e. new local notch stresses can be predicted at defined intervals in the longitudinal direction of the weld seam.

The modeling method can be used in the same way for different weld seam applications. FIG. 8 shows the possible applications for T-joints 9a, butt joints 9b and overlap joints 9c. For double-sided welded joints, one weld seam model is preferably used on each side. The effective notch stress prediction algorithm can preferably be fitted in such a way that it can be used unchanged for all these applications. For higher prediction accuracy, however, specialized effective notch stress prediction algorithms can also be fitted for individual applications.

As shown, for example, in FIGS. 7-8b and 8c, simple fillet welds involve welding components without geometric weld preparation. In order to obtain a better and continuous mechanical connection, the mechanical parts are also often connected as shown in FIG. 8a and thus provided with a geometric weld seam preparation. FIG. 4 shows a weld seam modelled in accordance with the invention in which the weld seam preparation on the component is fully modelled and meshed. However, as shown in FIG. 5, the components can also be modelled without weld seam preparation. This is made possible by independent meshing of the weld and the connection of the welds to the neighboring parts via FE coupling elements or coupling equations 7a. This facilitates the variation of the weld seam geometry without having to change the finite element model of the mechanical parts themselves.

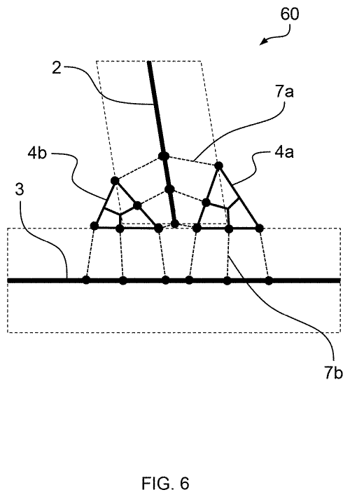

As shown in FIG. 6, the present modeling method also allows the application to shell models 60 in the same way, where components 2, 3 are meshed with finite shell elements in the center plane of the mechanical parts 2, 3 and welds 4a, 4b with the defined mesh pattern and the real weld geometry. The connection is again made with coupling elements or coupling equations 7a, 7b. The present modeling and notch stress prediction method can therefore be used in many different applications (FIG. 4, 5, 6, 8).

FIG. 9 shows a schematic flowchart of an execution example of a computer-implemented method for model generation and fatigue strength assessment of welds 4a, 4b between mechanical parts 2, 3 of an assembly using a finite element method. The procedure of FIG. 9 comprises the process steps S1 to S3 and is explained with reference to FIGS. 4 to 8:

In step S1, a finite element model 40 (see FIG. 4), 50 (see FIG. 5), 60 (see FIG. 6) is provided for the assembly. For finite element model 40, 50, 60 a first finite element mesh for a first mechanical part 2, a separate finite element mesh for a second mechanical part 3 and a third finite element mesh for a weld 4a, 4b connecting the first mechanical part 2 and the second mechanical part 3 having a number of notches 5a, 5b, 5c is created. The third finite element mesh has a number of less than 20 finite elements 6a, 6b, 6c in cross-section. The notches 5a, 5b, 5c of the weld 4a, 4b are modelled sharp-edged. The distribution of the finite elements follows a defined mesh pattern 8a, 8b, 8c (see FIG. 8). For example, the first finite element mesh and the third finite element mesh are coupled by a number of FE coupling elements 7a, 7b, 7c and the second finite element mesh and the third finite element mesh are coupled by a second number of FE coupling elements 7a, 7b, 7c.

In step S2, the finite element model 40, 50, 60 is calculated. From the defined mesh pattern 8a, 8b, 8c of the third finite element mesh for the welds 4a, 4b result values of the defined elements and nodes are provided.

For example, step S2 comprises the following substeps:
Calculating the finite element model 40, 50, 60, and
Evaluating the result values of the finite elements and the nodes of the defined mesh pattern 8a, 8b, 8c for the weld 4a, 4b on the basis of the calculated finite element model 40, 50, 60.

Preferably, result values are evaluated exclusively within the finite elements and nodes of the third finite element mesh for the weld 4a, 4b. The result values preferably comprise and consist of stress results, reaction force results, geometry parameters, and/or material parameters.

In step S3, an effective notch stress prediction algorithm is applied to predict occurring stresses in notches 5a, 5b, 5c using the provided result values as input parameters. The effective notch stress prediction algorithm predicts the occurring notch stresses in the notches 5a, 5b, 5c in their rounded state. The applied effective notch stress prediction algorithm is adapted to the defined mesh pattern 8a, 8b, 8c of the third finite element mesh.

The effective notch stress prediction algorithm is preferably trained before its application with a plurality of weld parameter variants for weld 4a, 4b using the defined mesh pattern 8a, 8b, 8c. By means of the effective notch stress prediction algorithm a plurality of parameters is predicted. This plurality of parameters comprises: principal stresses, shear stresses, Von Mises equivalent stresses, radial-, tangential- and/or axial-stress components in the notch radius of the respective notch 5a, 5b, 5c.

The predicted notch stresses can then be used to perform fatigue strength assessments of the assembly.

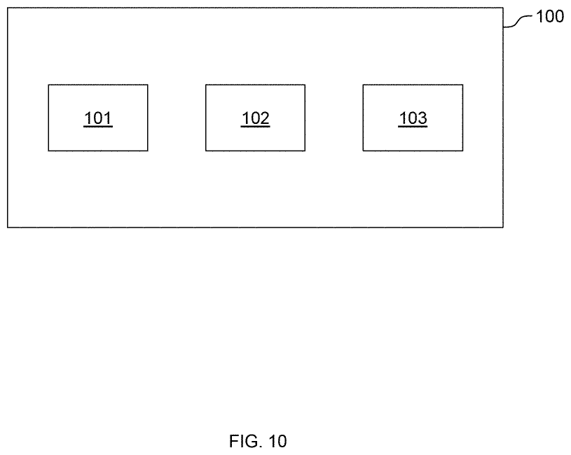

FIG. 10 shows a schematic block diagram of a design example of a computer-implemented device 100 for modelling and fatigue strength assessment of welds 4a, 4b between mechanical parts 2, 3 of an assembly using a finite element method.

The device 100 comprises a first unit 101, a second unit 102 and a third unit 103.

The first unit 101 is configured to provide a finite element model 40, 50, 60 for the assembly, in which a first finite element mesh for a first mechanical part 2, a separate second finite element mesh for a second mechanical part 3 and a third finite element mesh for a weld 4a, 4b connecting the first mechanical part 2 and the second mechanical part 3 comprising a number of notches 5a, 5b, 5c are generated. The third finite element mesh has a number of less than 20 finite elements 6a, 6b, 6c in the notches 5a, 5b, 5c of the weld 4a, 4b are sharp-edged and the distribution of the finite elements follows a defined mesh pattern 8a, 8b, 8c.

The second unit 102 is configured to calculate the finite element model 40, 50, 60, whereby 8a, 8b, 8c of the defined mesh pattern of the third finite element mesh are provided for the weld 4a, 4b result values of the finite elements and nodes.

The third unit 103 is configured to apply an effective notch stress prediction algorithm matched to the defined mesh pattern 8a, 8b, 8c of the third finite element mesh for predicting occurring notch stresses in the notches 5a, 5b, 5c using the provided result values as input parameters.

Although the present invention was described by means of design examples, it can be modified in many ways.

### LIST OF REFERENCE CHARACTERS

2 mechanical part
3 mechanical part
4 weld seam
4a weld seam
4b weld seam
5a notch
5b notch
5c notch
6a finite element
6b finite element
6c finite element
30 finite-element-model
7a coupling element
7b coupling element
7c coupling element
8a mesh pattern
8b mesh pattern
8c mesh pattern
9a T-joints
9b butt welds
9c lap joint
10 assembly
40 finite-element-model
50 finite-element-model
60 finite-element-model
100 device
101 first unit
102 second unit
103 third unit
S1 method step
S2 method step
S3 method step

### REFERENCES

[1] IIW Fatigue Recommendations: “Recommendations for Fatigue Design of Welded Joints and Components” from International Institute of Welding (IIW) A. F. Hobbacher
[2] FKM Guideline: “Analytical Strength Assessment of Components” from Forschungskuratorium Maschinenbau (FKM) (VDMA Verlag)
[3] CN103838975A
[4] DE102012023670A1
[5] DE102014224129A1
[6] EP1337942B1
[7] EP3267338A1
[8] JP2003080393A
[9] US2013325417A1

1. Computer-implemented method for model generation and fatigue strength assessment of weld seams between mechanical parts of an assembly with the aid of a finite element method, characterized by:
a) providing a finite element model for the assembly, in which a first finite element mesh for a mechanical part, a separate second finite element mesh for a second mechanical part and a third finite element mesh for a weld seam joining the first mechanical part and the second mechanical part comprising a number of notches are generated, wherein the third finite element mesh has a number of less than 20 finite elements in cross-section, the notches of the weld seam are modelled sharp-edged and the distribution of the finite elements follows a defined mesh pattern,
b) calculating the finite element model, wherein result values of the finite elements and nodes are provided from the defined mesh pattern of the third finite element mesh for the weld seam, and
c) applying an effective notch stress prediction algorithm matched to the defined mesh pattern of the third finite element mesh to predict occurring notch stresses in the notches using the provided result values as input parameters.

2. Method according to claim 1,
characterized in,
that the first finite element mesh and the third finite element mesh are coupled by means of a first number of coupling elements and the second finite element mesh and the third finite element mesh are coupled by means of a second number of coupling elements.

3. Method according to claim 1,
characterized in,
that step b) is formed by:
b1) calculating the finite element model, and
b2) evaluating the result values of the finite elements and the nodes of the defined mesh pattern for the weld seam on the basis of the calculated finite element model.

4. Method according to claim 3,
characterized in,
that in step b2) result values are exclusively evaluated within the finite elements and the nodes of the third finite element mesh for the weld seam.

5. Method according to claim 3,
characterized in,
that the result values, which are evaluated in step b2), include
stress results,
reaction force results,
geometry parameters, and/or
material parameters.

6. Method according to claim 3,
characterized in,
that the result values, which are evaluated in step b2), consist of
stress results,
reaction force results,
geometry parameters, and/or
material parameters.

7. Method according to claim 1,
characterized in,
in that the effective notch stress prediction algorithm is trained with a plurality of weld seam parameter variants using the defined mesh pattern before the application of step c)

8. Method according to claim 1,
characterized in,
in that in step c) a plurality of parameters of the notches are predicted by means of the effective notch stress prediction algorithm.

9. Method according to claim 8,
characterized in,
that the parameters include:
normal stresses,
shear stresses,
Von Mises equivalent stresses,
radial-, tangential stress components, and/or
axial stress components in the notch radius of the respective notch.

10. Method according to claim 1,
characterized in,
that the predicted notch stresses are then used to perform fatigue strength assessments of the assembly.

11. Computer program product which, on a program-controlled device, initiates the execution of the method according to claim 1.

12. Computer-implemented device for modelling and fatigue strength assessment of weld seams between mechanical parts of an assembly with the aid of a finite element method, characterized by:
a first unit for providing a finite element model for the assembly, in which a first finite element mesh for a first mechanical part, a separate second finite element mesh for a second mechanical part and a third finite element mesh for a weld connecting the first mechanical part and the second mechanical part comprising a number of notches, wherein the third finite element mesh comprises a number of less than 20 finite elements in cross-section, wherein the notches of the weld seam are modelled with sharp edges and the distribution of the finite elements follows a defined mesh pattern,
a second unit for calculating the finite element model, wherein result values of the finite elements and nodes are provided from the defined mesh pattern of the third finite element mesh for the weld seam, and
a third unit for applying an effective notch stress prediction algorithm matched to the defined mesh pattern of the third finite element mesh to predict occurring notch stresses in the notches using the provided result values as input parameters.
